(19) United States
(12) Patent Application Publication
(10) Pub. No.: US 2024/0066321 A1
(43) Pub. Date: Feb. 29, 2024

(54) A MULTIPLANAR MOTION MANAGEMENT SYSTEM

(71) Applicant: EBAMed SA, Geneva (CH)

(72) Inventors: Saskia CAMPS, Prangins (CH); Jérémie GRINGET, Lausanne (CH); Rosalind PERRIN, Satigny (CH); Adriano GARONNA, Geneva (CH)

(21) Appl. No.: 18/269,351

(22) PCT Filed: Dec. 23, 2021
(86) PCT No.: PCT/IB2021/000922

§ 371 Date: Jun. 23, 2023

Related U.S. Application Data
(60) Provisional application No. 63/129,694, filed on Dec. 23, 2020.

(51) Int. Cl. A61N 5/10

(52) U.S. Cl. CPC A61N 5/1068; A61B 5/33

(57) ABSTRACT

A multiplanar motion management system to aid in administering non-invasive radiation therapies. The disclosed devices and techniques enable clinicians to accurately monitor the position and motion of the heart with respect to a reference situation of the simulation stage. In addition, information is provided about the respiratory phase of the patient as well as about possible drifts of the anatomical structures. This information can be used by the clinician to interrupt the radiation treatment manually when necessary. The information can also be used to automatically, and in real-time, provide the appropriate gating signals to the therapy machine. The disclosed system may be configured to cover all motion aspects required during a typical radiotherapy workflow with just one device, from patient setup (inter-fraction motion monitoring) to cardiac and respiratory motion monitoring during the treatment (intra-fraction motion monitoring).

### RELATED APPLICATIONS

This patent application claims the benefit of U.S. Provisional Patent Application No. 63/129,694, filed Dec. 23, 2020, the disclosure of which is hereby incorporated by reference in its entirety.

### FIELD OF THE DISCLOSURE

This disclosure is directed generally to devices and techniques for external beam therapy, and more specifically to non-invasive treatment of cardiac arrhythmias.

### BACKGROUND OF THE DISCLOSURE

Cardiac arrhythmias are disruptions in the normal heartbeat. They affect more than two percent of the general population in Europe and are expected to at least double in the next 50 years as the population ages. Their occurrence is strongly linked to an increased risk of heart attacks and strokes.

More particularly, heart arrhythmia is a problem with the rate or rhythm of the heartbeat. The heart beats too quickly, too slowly, or with an irregular pattern. When at rest, the heart beating faster than normal, above 100 beats per minute in adults, is called tachycardia. The heart beating too slowly, below 60 beats per minute, is called bradycardia. A common type of arrhythmia is atrial fibrillation, which causes an irregular and fast heartbeat. Many factors can affect the rhythm of the heart such as having had a heart attack, smoking, congenital heart defects, and stress. Some substances or medicines may also cause arrhythmias.

Treatments may include medications, medical procedures such as ablation therapy or implantation of a pacemaker or defibrillator, and surgery. Medications for a fast heart rate may include beta-blockers or agents that attempt to restore a normal heart rhythm such as procainamide. Medications may have more significant side effects especially if taken for a long period of time. Pacemakers are often used for slow heart rates. Those with an irregular heartbeat are often treated with blood thinners to reduce the risk of complications. Those who have severe symptoms from an arrhythmia may receive urgent treatment with a controlled electric shock in the form of cardioversion or defibrillation.

Ablation therapies are often used to treat arrhythmias. Ablation therapies include burning or freezing specific heart tissues with invasive tools such as catheters, to stop the conduction of the disrupted electrical signals in these specific tissues. Invasive catheter ablation procedures are surgical interventions performed manually and the treatment efficacy varies largely from 50% to 80% depending on the technology used and skill of the surgeon. Moreover, the procedures can involve several medical staff members and can require many hours, during which patients are at risk of serious complications like tissue perforation, vein stenosis, or creation of a blood clot. The nature of the lesions created by catheter ablation results in procedures which are often repeated successive times, with increasing complexity for the medical staff and risk for the patient.

There is therefore a need for methods of treatment using non-invasive devices for treatment of cardiac arrhythmias via non-invasive ablation. The use of external therapy beams, including but not limited to charged particle beams, photon beams, proton beams, and carbon and helium ions, offer non-invasive techniques for cardiac ablation. Improvement of devices and methods for such external beam therapies that take into account body motion of the patient in real would be welcomed.

### SUMMARY OF THE DISCLOSURE

Various embodiments of the disclosure enable an accurate setup of patients and beam delivery for external therapy beams based on the position of the patient's heart. This can be beneficial not only for cardiology patients, but also, for example, for lung cancer patients undergoing radiotherapy where the heart might be an organ at risk that should be tracked during the treatment. The disclosed devices and techniques expand upon International Patent Application No. PCT/US2020/060054 to Garonna et al. (“Garonna et al.”), entitled “Cardiac Phase Gating System for Radiation Therapy,” filed Nov. 11, 2020 and owned by the owner of the present application. The present disclosure expands on Garonna et al. by providing an improved method for identifying the respiratory motion phase and to determine an absolute heart position in a known coordinate system in three dimensional space. Various embodiments of the disclosure track the heart motion without the segmentation of any anatomical landmarks (or fiducials) in the image to determine the target motion phase and position. Such techniques also do not require the co-registration or fusion of ultrasound with MRI or CT images, thereby avoiding the inaccuracies and execution time requirements associated with registration across different modalities. The presently disclosed devices and techniques are therefore advantageous in that the image processing and motion measurement is fully automatic (no operator or clinician involvement), reproducible, and faster than conventional motion control systems.

In some embodiments, external motion of the patient's thorax as detected with a surrogate is combined with the internal motion of the heart as detected by a multiplanar probe that generates multiplanar (non-parallel) images to better understand how the heart moves due to respiratory motion. The combination of external and internal motion detection enables extraction of a respiratory motion pattern from images of the heart. The devices and techniques disclosed are further able to determine the position of cardiac structures in a known three-dimensional coordinate system by a combination of multiplanar images and a probe localization system. In addition, the techniques disclosed are robust enough to detect and quantify using images the heart drift that may occur during external beam therapy.

Some embodiments provide robustness in terms of image quality (e.g., ultrasound) by enabling imaging from different thorax positions (in particular, parasternal and apical viewing windows) depending on the patient's anatomy. Also the image processing methods are designed with image quality robustness in mind. This can be beneficial for patients of advanced age or having higher body mass index, common for arrhythmia patients, which can lead to ultrasound images of less contrast due to a low echogenicity.

Various embodiments of the disclosure include redundancies which are inherently part of the design. For example, in the case of cardiac-phase recognition failure in the imaging system, the cardiac phase-gating can still be performed using the ECG signal. In another example, a localization marker on the multiplanar probe may be utilized to track external motion to enable respiratory phase gating in case of such failure in the imaging system.

Various embodiments of the disclosure enhance alignment of a treatment patient with the particle beam. Standard clinical practice involves alignment using bony anatomy. In some embodiments, this bony anatomy alignment is complemented with a heart alignment setup using ultrasound imaging.

Various embodiments of the disclosure present a system and methods for cardiac ultrasound three-dimensional motion management. A multiplanar ultrasound probe is utilized and dedicated image processing methods are disclosed for determining the position and motion of the heart in three dimensions in an accurate and fast fashion before and during external beam therapy. In some embodiments, a method is disclosed that enables accurate patient positioning based on the heart position, monitoring of potential drift in heart position during a long treatment session and monitoring of respiratory motion during treatment.

The disclosed device and methods are beneficial for the non-invasive treatment of heart arrhythmias. However, its use can also apply favorably for cancer treatments in the mediastinum and thoracic regions (breast, lung, among others), as in these cases, the precision of the treatment is affected by the position and motion of the heart, especially in the case of pencil-beam scanned particle therapy. Indeed, for these clinical applications, the heart is not the target but an important sensitive organ that clinicians want to spare, to avoid serious side-effects related to radiation.

The disclosed device and methods can also be applied outside the field of external beam radiotherapy, for example, in radiology and interventional cardiology. For radiology, it can provide means to obtain better image quality using magnetic resonance imaging (MRI), computed tomography (CT) or other medical imaging modalities, for which the heart motion (due to respiration or heartbeat) can affect the image quality and therefore influence the derived measurements and diagnosis. This would allow for, for example, avoiding the use of breath-hold or forced ventilation techniques to limit respiratory motion. For interventional cardiology, it can provide means to monitor the heart motion during the intervention. This enables the interventional cardiologist to acquire, for example, X-ray images used to visualize a catheter only at a specific cardiac or respiratory state of interest (motion phase). This would allow for a more efficient use of X-ray imaging, which reduces the radiation dose delivery to both the patient and medical staff as well as operational costs.

In some embodiments, the disclosed device and methods utilize B-mode ultrasound imaging. However, other imaging techniques are contemplated, for example, Doppler, Elastography, M-mode imaging and several other ultrasound imaging modalities. Also, the present disclosure is based on biplane ultrasound imaging that utilizes orthogonal planes; however, one of ordinary skill in the art, in view of this disclosure, may incorporate and adapt imaging techniques more generally, for example, biplane images which are not orthogonal, triplane imaging, full volumetric imaging or any other multiplanar ultrasound imaging geometry in three-dimensional space.

Conventional systems and procedures for the augmentation and execution of external beam radiation therapy treatments (radiotherapy, proton therapy, carbon ion therapy, among others) are subject to certain shortcomings. Examples where shortcomings are limiting in present day systems include patient setup, organ drift during treatment, accounting for respiratory motion, and tracking anatomical structures in three-dimensional space.

Consider the challenges of patient positioning for external beam radiation therapy treatment of the heart. Prior to the start of a course of external beam radiation therapy treatment, a computed tomography (CT) scan of the patient is typically acquired at the so-called simulation stage. Subsequently, a patient-specific treatment plan is designed based on this CT scan, after which the treatment stage can start, but generally not on the same day. During the treatment stage, the position of the patient during the simulation stage is reproduced in the treatment room to ensure the validity of the treatment plan and the beam parameters programmed for the treatment delivery. Immobilization devices, such as thermoplastic masks, standard-sized wedges, wing boards and grab bars, are typically used to assist with patient positioning for thoracic treatments. Technologies currently available to assist with this patient setup include bi-planar X-ray images, cone-beam CT or CT-on-rails. The patient alignment in the treatment room is mostly based on matching of bony anatomy (both using manual and (semi-) automatic image registration) and matching of any soft tissue close to the target which has sufficient contrast on these images (e.g., tomographic imaging). The contrast of soft tissues such as the heart on these images is typically poor. Also, the position of the heart does not always correlate well with the bony anatomy. In addition, the heartbeat and the respiratory motion of the patient further reduce the image quality. The image quality can also be affected by image artifacts created by metallic objects commonly present in the area of interest (pacemaker leads, implanted defibrillator leads, among others). Accordingly, the setup of arrhythmia patients and thorax patients in general suffers from the limited anatomical information available on the acquired images and consequently relies heavily on the judgment of the medical staff members.

Consider also the phenomenon and effect of heart drift. During external beam therapy treatment of heart arrhythmias, a radiation dose in the range of 25-40 Gy may be delivered in a single treatment fraction. This is at least 2-3 times the typical stereotactic ablative radiotherapy dose for small thoracic lesions. In addition, special methods may be used during the treatment to mitigate the effect of respiratory and cardiac motion on the treatment accuracy. Both aspects (high dose requirement and motion mitigation) result in considerably longer treatment times, with the patient typically immobilized in supine position. As such, anatomical structures in the thorax/abdomen relax, which can result in a positional drift of the target over time, herein referred to as “heart drift.” While methods are emerging for efficient intra-fraction motion management, such as the MR-LINAC, there is no standard method to monitor this drift during the treatment without interrupting the radiation beam delivery, which increases the treatment time even further.

The effects of the respiratory cycle must also be managed. The cyclical heart motion is characterized by two primary components: heartbeat (with cycle period 0.5-1.5 seconds) and respiration (with cycle period of 3-5 seconds). Previous work directed to such characterization includes Garonna et al., which outlined the respiratory component but was focused on heartbeat motion aspects. For respiratory motion monitoring, systems are commercially available which indirectly monitor the internal motion of anatomical structures such as the heart by following external surrogates. These systems are either based on the flow of air into and out of the lungs (e.g., with spirometers, such as utilized by the Active Breathing Coordinator, or “ABC,” device supplied by Elekta AB, Stockholm, Sweden) or the mechanical motion of the abdomen/thorax resulting from contraction of the diaphragm, such as pressure belts (e.g., the ANZAI BELT (AZ-733VI) supplied by Anzai Medical Co., Ltd. of Tokyo, Japan), optical tracking of thorax motion (e.g. the RGSC system supplied by Varian Medical Systems, Inc. of Palo Alto, California, U.S.A.), and x-ray tracking systems (e.g., the tracking system in VERO® SBRT, supplied by Brainlab AG of Munich, Germany). These approaches make assumptions about how the motion of the surrogate correlates with the internal motion of the anatomical structures and, thus, also of the behavior of the treatment target. If this correlation is weak, this may result in a poor monitoring accuracy of the target respiratory motion. Robotic radiosurgery systems (e.g., the CYBERKNIFE®, supplied by Accuray Inc. of Sunnyvale, CA, U.S.A.) are able to manage intra-fractional respiratory motion. However, its use for the heart would require implanting fiducial clips.

Several approaches are described in literature which facilitate the tracking of a treatment target on three-dimensional ultrasound volumes, which have been acquired of a specific anatomical structure or region of interest. Examples of clinically available systems are the CLARITY® and the CLARITY® AUTOSCAN systems (supplied by Elekta AB, Stockholm, Sweden). In certain applications, tracking a target in three-dimensional space requires volumetric imaging. Such requirement arises, for example, when the target can move out of the imaging plane, which would not necessarily be detected with two-dimensional imaging, thereby resulting in faulty tracking. However, tracking a target in three-dimensional space on three-dimensional ultrasound volumes suffers from a number of limitations:
Existing commercial and research grade three-dimensional ultrasound systems have inadequate spatial and time resolution.
There is a need, sometimes manually, to identify the target or a surrogate of the target whose motion correlates well with the target motion in the ultrasound volume
Three-dimensional ultrasound imaging technology is bulky and expensive.
   We estimate that a system comprised of conventional technology for tracking anatomical structures inside the heart in three-dimensional space would have, at best, the following performance characteristics:
Reaction time (time between the appearance of an event and the final response of the system) would be about 250 milliseconds (ms). This is composed of 200 ms for three-dimensional ultrasound volume acquisition, beam forming and data streaming from the ultrasound device to a host computer and 50 ms for data interpretation.
Spatial uncertainty for the target localization is about 6.5 mm, a combination of: 1 mm for ultrasound image localization in absolute room coordinates; 2.5 mm for segmentation and tracking accuracy of a target or a surrogate where the target itself cannot be properly tracked; 2 mm for motion correlation between the target and the surrogate of the target; and 1 mm for anatomical structure visualization differences between the planning CT scan and the ultrasound volume.
   Such performance characteristics are inadequate for external radiotherapy treatment of the heart, where a reaction time of 100 ms or better and a spatial accuracy of 3 mm is expected.

The present disclosure presents hardware and software components and methods for overcoming certain present-day limitations. In addition, various clinical workflows are also disclosed.

In some embodiments, an ultrasound imaging probe is disclosed that provides non-invasive image acquisition in real-time without the use of ionizing radiation which could potentially harm the patient. In addition, it is cost effective, it typically provides good soft tissue contrast and it is often the diagnostic tool of choice for cardiology patients.

Structurally, various embodiments of the disclosure include multiplanar motion management system for non-invasive cardiac ablation comprising: a multiplanar imaging probe configured to acquire live multiplanar images within a probe volume, the multiplanar images being non-parallel; a probe localization system configured to acquire positional data of the multiplanar imaging probe; an input/output (I/O) management system configured to concurrently receive the live multiplanar images and the positional data, and to establish acquisition times for the multiplanar images and for the positional data; and a gating system configured to receive and process the live multiplanar images, the positional data, and the acquisition times. The gating system includes a processor that may be configured to: identify a cardiac phase corresponding to the live multiplanar images; define a set of reference coordinates from the positional data that closest corresponds in time to the live multiplanar images; and perform a rigid registration of the live multiplanar images to reference multiplanar images, the reference multiplanar images being representative of the cardiac phase. Some embodiments include a heartbeat sensing system for generating cardiac cycle data, wherein: the heartbeat sensing system is configured to generate a trigger signal based on the cardiac cycle data; the I/O management system is configured to receive the trigger signal concurrently with the multiplanar images and the positional data, and to generate trigger signal information that includes an acquisition time corresponding to the trigger signal; and the gating system is configured to receive and process the trigger signal information for identification of a start of a cardiac cycle. The gating system may be configured to utilize the trigger signal information to confirm the cardiac phase. In some embodiments, the heartbeat sensing system is an electrocardiogram (ECG) system and the cardiac cycle data is generated from an electrocardio signal. The positional data may be relative to a reference coordinate system, and the positional data may localize a location and orientation of the multiplanar imaging probe in three-dimensional space. The multiplanar imaging probe may be configured to acquire the multiplanar images simultaneously, and may include a geometry configured to mount on the thorax of a patient. In some embodiments, the multiplanar imaging probe acquires the multiplanar images at a rate that is in a range of 20 Hz to 50 Hz inclusive. In some embodiments, the multiplanar imaging probe is a multiplanar ultrasonic probe and the reference multiplanar images are ultrasonic images.

Various embodiments of the disclosure embody a method, comprising: (a) acquiring a series of reference multiplanar images of a beating heart within a patient with a multiplanar imaging probe; (b) during step (a), acquiring a reference position of the imaging probe for each of the reference multiplanar images of the reference series of multiplanar images; (c) identifying a cardiac phase for each reference multiplanar image of the reference series of multiplanar images; (d) acquiring a live multiplanar image with the multiplanar imaging probe during beam therapy treatment of the patient; (e) during step (d), acquiring a live position of the multiplanar imaging probe; (f) identifying a cardiac phase of the live multiplanar image; and (g) performing a rigid registration of the live multiplanar image and the live position to a corresponding reference image of the reference series of multiplanar images and a corresponding reference positions associated with the corresponding reference image, the corresponding reference image being representative of the cardiac phase of the live multiplanar image, the rigid registration providing a live heart position of the beating heart during the beam therapy treatment of the patient relative to a three-dimensional coordinate system to provide a live displacement of the beating heart between relative to the reference position.

In some embodiments, method includes a step of configurating a tangible, non-transitory medium the enumerated steps as instructions. The instructions in the step of configuring includes generating a status condition based on a comparison of the displacement with a predetermined threshold value. The method may also include affixing the multiplanar imaging probe to a thorax of the patient for use during execution of a reference simulation. In some embodiments, the method includes affixing the multiplanar imaging probe to a thorax of the patient for use during execution of a simulation stage. The method may include instructing the patient to breath hold over at least one cardiac cycle to effect the known respiratory state. The known respiratory state may be an end-expiration respiratory phase. The reference series of multiplanar images may be acquired at the start of a treatment process. In some embodiments, the reference series of multiplanar images is acquired during a simulation stage prior to a treatment stage.

In some embodiments, the method includes coupling a multiplanar imaging probe and a probe localization system to a gating controller, the gating controller including the set of instructions preconfigured on a non-transitory computer readable medium for execution in real time. The instructions of the gating controller may include generating a status condition based on a comparison of the displacement with a predetermined threshold value. In some embodiments, the method includes configuring the non-transitory computer readable medium with the instructions.

Various embodiments of the disclosure include a method for determining a position of cardiac structures in a known three-dimensional coordinate system, comprising: providing a multiplanar imaging probe and a localization system for tracking the multiplanar imaging probe in three-dimensional space; providing instructions on a tangible, non-transitory medium. The instructions may include: acquiring a first sequence of multiplanar images of a heart with the multiplanar imaging probe over a duration of multiple heartbeats while the patient is in a known respiratory state, the heart being at a first position; during the step of acquiring the first sequence, localizing the multiplanar imaging probe with the localization system to define a first set of reference coordinates at the first position; acquiring a second sequence of multiplanar images of the heart with the multiplanar imaging probe over a duration of multiple heartbeats while the patient is in the known respiratory state, the heart being at a second position; during the step of acquiring the second sequence, localizing the multiplanar imaging probe with the localization system to define a second set of reference coordinates at the second position; identifying a first set of representative multiplanar images from the first sequence of multiplanar images and a second set of representative multiplanar images from the second sequence of multiplanar images, the first set of representative multiplanar images and the second set of representative multiplanar images corresponding to a known combination of a cardiac motion phase and a respiratory motion phase; aligning the second set of reference coordinates with the first set of reference coordinates; and quantifying a translational displacement and a rotational displacement of the second set of representative multiplanar images relative to the first set of representative multiplanar images within the aligned first and second set of reference coordinates. In some embodiments, the known combination of cardiac motion phase and respiratory motion in the step of identifying correspond with an end-expiration of the respiratory motion phase. The instructions in the step of providing instructions may include displacing a patient support in accordance with the translational displacement and the rotational displacement. In some embodiments, the instructions in the step of providing instructions include instructing the patient to breath hold over at least one cardiac cycle during the step of acquiring the first sequence of multiplanar images to effect the known respiratory state. The step of acquiring the first sequence of multiplanar images of the instructions may be performed at an end-expiration respiratory phase to effect the known respiratory state.

Various embodiments of the disclosure include a method for inferring displacement of the heart resulting from respiratory motion from a multiplanar imaging probe mounted to a thorax of a patient, comprising: providing a multiplanar imaging probe; providing instructions on a tangible, non-transitory medium. The instructions comprise: acquiring a reference series of multiplanar images with the multiplanar imaging probe, the multiplanar imaging probe being affixed to a thorax of a patient; identifying a cardiac phase for each multiplanar image of the reference series of multiplanar images; acquiring a live multiplanar image with the multiplanar imaging probe during irradiation treatment of the patient, the multiplanar imaging probe being affixed to the thorax of the patient; identifying a cardiac phase for the live multiplanar image; and performing a rigid registration of the live multiplanar image to a corresponding image of the reference series of multiplanar images, the corresponding image of the reference series being representative of the cardiac phase of the live multiplanar image.

### BRIEF DESCRIPTION OF THE DRAWINGS

FIGS. 1 and 2 are perspective views of an ultrasound probe for acquiring multiplanar images in two non-parallel imaging planes according to an embodiment of the disclosure;

FIG. 3 is a simplified perspective view of a non-invasive cardiac ablation system system according to an embodiment of the disclosure;

FIG. 4 is a schematic of the non-invasive cardiac ablation system of FIG. 3 according to an embodiment of the disclosure;

FIG. 5 is a workflow diagram illustrating a simulation stage for development of a treatment plan according to an embodiment of the disclosure;

FIG. 6 is a workflow diagram illustrating a treatment stage for delivering radiation therapy according to an embodiment of the disclosure;

FIGS. 7 through 9 illustrate the alignment of treatment stage images with simulation stage images according to an embodiment of the disclosure;

FIG. 10 is a plot depicting live-to-simulation registration and localization characteristics of heart displacement for analysis of respiratory cycle and heart drift according to an embodiment of the disclosure; and

FIG. 11 is a flow chart of a method for extracting respiratory cycle and heart drift information of FIG. 10 according to an embodiment of the disclosure.

### DETAILED DESCRIPTION

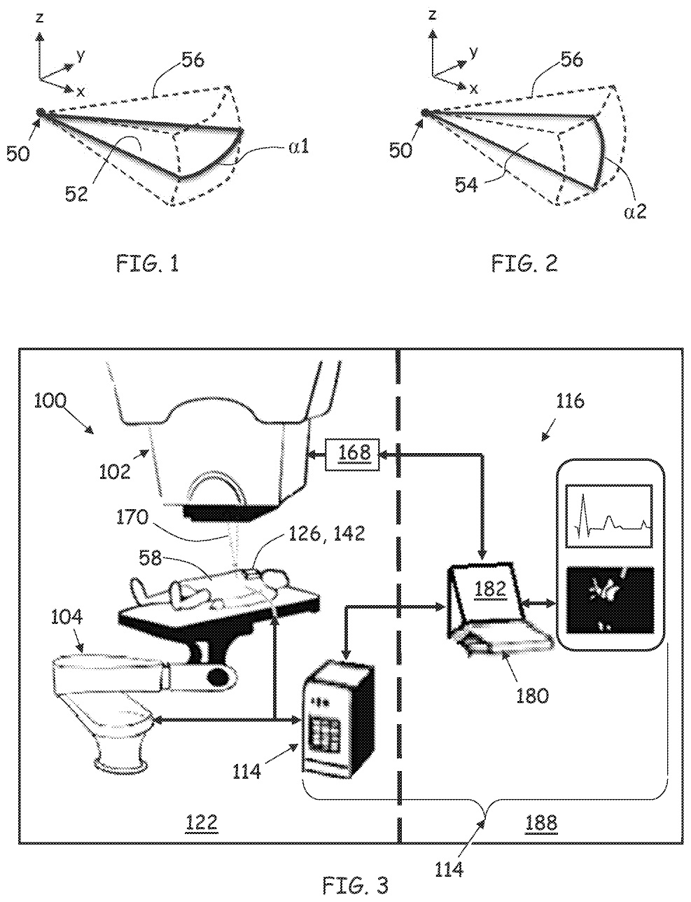

Referring to FIGS. 1 and 2, the action of a multiplanar ultrasound probe 50 is depicted according to an embodiment of the disclosure. The multiplanar ultrasound probe 50 is configured to acquire images in two non-parallel imaging planes 52 and 54. In some embodiments, the non-parallel imaging planes 52 and 54 are orthogonal to each other, referred to arbitrarily as the x-y plane (FIG. 2) and the x-z plane (FIG. 3). The multiplanar ultrasound probe 50 includes a two-dimensional array of sensors (not depicted) that may be scanned to provide an effective probe volume 56. The multiplanar ultrasound probe 50 may image each of the multiplanar images with resolution characteristics similar to single two-dimensional ultrasound imaging. Such multiplanar ultrasound probes are commercially available, for example the XMATRIX® X5-1 or X7-2t array transducer, provided by Koninklijke Philips Electronics N.V. of Eindhoven, Netherlands.

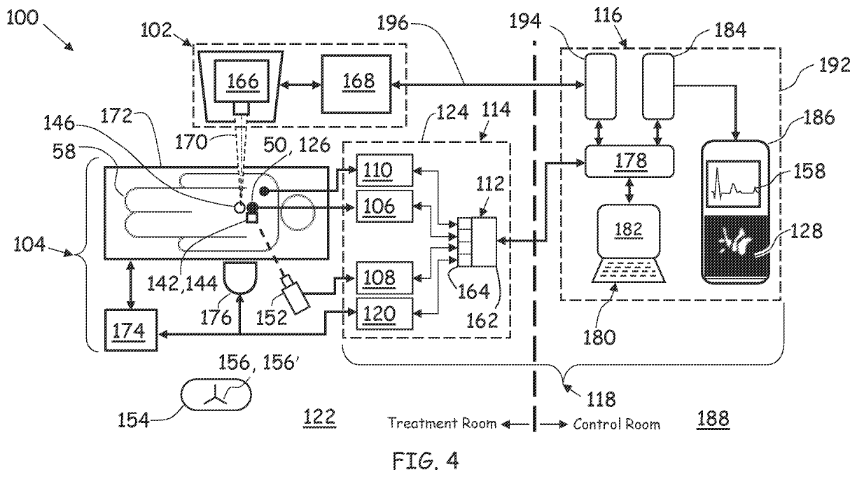

In some embodiments, the design of the probe is adapted to reduce the number of metallic components inside its casing, which reduces the appearance of artifacts on, for example, CT or cone beam CT imaging. The multiplanar ultrasound probe 50 may have a geometry (not depicted) which is tailored for positioning on the thorax of a patient 58 (FIG. 4). In some embodiments, the multiplanar images are acquired at an image refresh rate that is in a range of at least 10 to 30 Hertz inclusive. In some embodiments, the image refresh rate is in a range of 10 to 50 Hertz inclusive. In some embodiments, the image refresh rate is in a range of 10 to 100 Hertz inclusive. Herein, a range that is said to be “inclusive” includes the end point values of the stated range.

Functionally, the multiplanar ultrasound probe 50 enables live acquisition of non-parallel images (FIG. 4) simultaneously or in sequence, wherein “live” is defined as real time or near-real time acquisition sufficient for image monitoring. The non-parallel images acquired with the multiplanar ultrasound probe 50 enable monitoring of the heart motion in three-dimensional spatial coordinates within the effective probe 56. The non-parallel imaging planes 52 and 54 provide information in three dimensions about the imaged anatomical structures.

Referring to FIGS. 3 and 4, a non-invasive cardiac ablation system 100 is depicted according to an embodiment of the disclosure. The non-invasive cardiac ablation system 100 includes a therapy beam emitting system 102, a patient positioning system 104, a multiplanar imaging system 106, a probe localization system 108, and a heartbeat sensing system 110. The systems 106, 108, and 110 are controlled by a real time data acquisition system or DAQ 112, all of which are part of an input/output (I/O) management system 114. The I/O management system 114 interfaces with a beam gating system 116 to present a multiplanar motion management system 118. The I/O management system 114 may also include auxiliary interface(s) 120 for interfacing with other components of the non-invasive cardiac ablation system 100, for example the patient positioning system 104.

The I/O management system 114 concurrently acquires data sets from different data streams and establishes the time of acquisition (“time stamps”) corresponding to each data set. In some embodiments, the I/O management system 114 includes components for the acquisition of three types of data: ultrasound data, electrocardiogram (ECG) data, and optical localization data. The I/O management system 114 may be physically located in a treatment room 122 and, in some embodiments, housed or based in an I/O cabinet 124.

The multiplanar imaging system 106 is coupled to a multiplanar imaging probe 126 (e.g., multiplanar ultrasound probe 50) for generation of non-parallel multiplanar images 128 in real time or near real time for visualization of the heart. The multiplanar imaging system 106 may include the multiplanar ultrasound probe 50, a probe holder 142, and a beam former (not depicted). The beam former generates timed electrical signals that sends the ultrasound wave into the patient, collects the electrical signals coming from the probe elements based on the reflection in the patient of the acoustics waves, and processes the incoming signals in order to generate an ultrasound image.

The multiplanar images 128 may resolve a target region 146 directly or otherwise enable inference of the position of the target region 146. The multiplanar imaging probe 50, 126 and/or probe holder 142 may include or be fitted with one or more localization markers 144. The multiplanar imaging system 106 may be configured to withstand some level of radiation exposure, such as indirect emission of neutrons, protons and gamma rays from the proximate particle beam. Alternatively, some components of the multiplanar imaging system 106 (e.g., data processor) can be remote and signals transmitted via analog or digital transmission cables.

Use of the multiplanar ultrasound probe 50 as the multiplanar imaging probe 126, prevalent throughout this disclosure, is non-limiting. The skilled artisan, in light of this disclosure, will recognize that the concepts disclosed herein are not limited to ultrasound imaging, but may incorporate other non-parallel imaging devices and techniques that may be incorporated mutatis mutandis. Other multiplanar imaging systems 106 and attendant multiplanar imaging probes 126 are contemplated for the non-invasive cardiac ablation system 100, for example x-rays, MRI, and ECGI systems.

The probe localization system 108 may be an optical system, for example, a camera 152 fixed to view the localization markers 144. In one embodiment, the camera 152 is an infrared (IR) camera, such as the FUSIONTRACK® 500 available from Atracsys, SARL of Puidoux, Switzerland, or the POLARIS VEGA® XT available from Northern Digital Inc. of Waterloo, Canada. This type of camera can localize infrared-reflective or LED markers in 6 degrees of freedom in three-dimensional space, for example in the form of positional data that establishes the location and rotational orientation of the marker relative to the camera 152. Alternatively, the probe localization system 108 may operate by other principles, including electrical, magnetic, or acoustic. The probe localization system 108 may also include surface tracking capabilities, for example using lasers or thermal imaging.

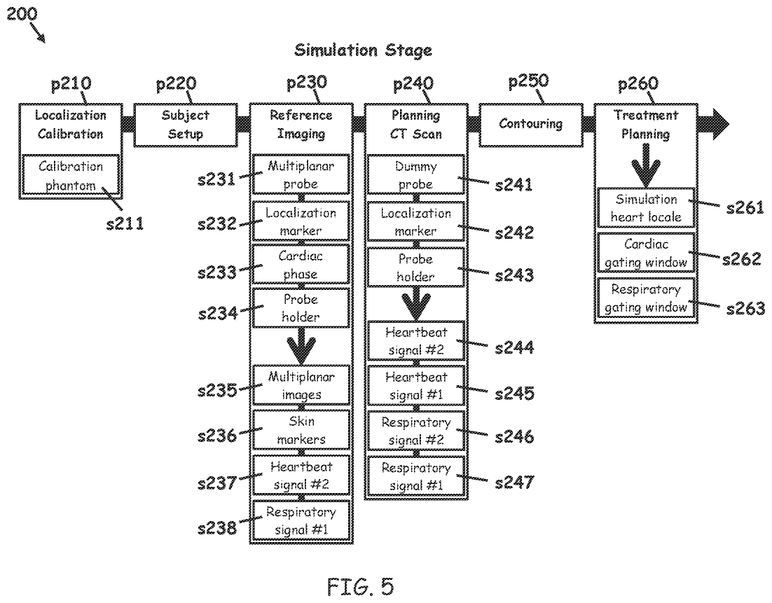

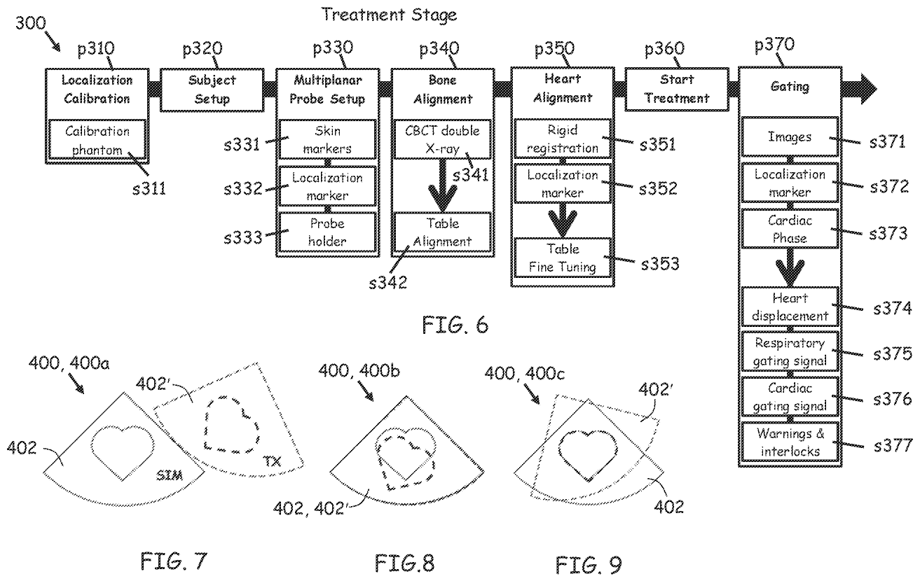

In some embodiments, the localization marker(s) 144 are positioned in a CT room (not depicted) during a simulation stage 200 (FIG. 5) and in the treatment room 122 during a treatment stage 300 (FIG. 6). The CT and treatment rooms may include a common room or reference coordinate system 154 for generating reference pixel coordinates 156′, 156 relative to a treatment isocenter (not depicted) of the non-invasive cardiac ablation system 100, where reference pixel coordinates 156′ refer to the simulation stage 200 and reference pixel coordinates 156 refer to the treatment stage 300. A calibration procedure with a dedicated calibration phantom (not depicted) may be used so that the probe localization system 108 provides a position and orientation of the multiplanar imaging probe 50, 126 in a congruent room coordinate system 154. Such calibration phantoms are known in the art, for example as used by Lachaine et al., “Intrafractional prostate motion management with the Clarity Autoscan system,” Medical physics international, 1 (2013).

The heartbeat sensing system 110 generates cardiac cycle data 158, such as an ECG measuring the electrical activity generated by the heart. The heartbeat sensing system 110 monitors heartbeat signal acquisition and outputs a trigger signal that is received by the I/O management system 114. In some embodiments, the I/O management system is configured to receive the trigger signal concurrently with the multiplanar images 128. For ECG systems, both amplitude and R-peak triggers of an electrocario signal may be monitored. An ECG system outputs the trigger pulse upon detection of the R-peak, the trigger pulse being received and time stamped by the I/O cabinet. The ECG system may also control the switching between ECC leads.

The DAQ 112, which controls at least the systems 106, 108, and 110, may include a programmable logic controller (PLC) 162 and various I/O modules 164. The PLC 162 and I/O modules 164 execute the start and stop triggering as well as the accurate time-stamping of the imaging, heartbeat, and localization data streams.

The therapy beam emitting system 102 and patient positioning system 104 are optionally coupled to the I/O management system 114. The therapy beam emitting system 102 may include a particle emitter 166 and a beam controller 168 for selectively generating a particle beam 170 which is delivered to the target region 146. The patient positioning system 104 may include a patient support 172 and a positioner system 174. In some embodiments, the non-invasive cardiac ablation system 100 includes a patient position verification system 176 which may also be coupled to the I/O management system 114. The patient position verification system 176 may include a double X-ray or a cone-beam computed tomography (CBCT) imaging system for verification of the patient positioning. Additional details of the therapy beam emitting system 102, patient positioning system 104, and the patient position verification system 176 are available at Garonna et al.

The beam gating system 116 includes hardware control and signal capabilities that are coupled to a gating controller 178. The target motion management system 118 may include a control console 180 for user interface with the gating controller 178. The control console may include a graphical user interface (GUI) 182 to facilitate user input and information display. The gating controller 178 is operatively coupled to receive input from and/or send output to the therapy beam emitting system 102, the patient positioning system 104, the multiplanar imaging system 106. The gating controller 178 may be interfaced with the I/O management system 114 via the DAQ 112. In addition to controlling and coordinating the hardware components of the non-invasive cardiac ablation system 100, the gating controller may include computer memory for storage and execution of the various computational tasks required of the beam gating system 116, including but not limited to registration of multiplanar images, determination of cardiac phase (e.g., pursuant to Garonna et al.).

The beam gating system 116 processes the data and generates updated information to the user and to the non-invasive cardiac ablation system 100. The beam gating system 116 may be used for real time or near real time processing of the image, heartbeat, and localization data streams, for example, using a graphics processing unit (GPU) 184. Alternatively, the GPU 184 may be located in the treatment room 122 and housed in the I/O cabinet 124 as part of the I/O management system 114. The GPU 184 may be in communication with the GUI 182 and auxiliary display monitors 186 that, for example, display data such as the multiplanar images 128 and the cardiac cycle data 158. The beam gating system 116 may also receive information from the operator through the GUI 184. The beam gating system 116 may be remotely located in a control room 188 separate from the treatment room 122 and, in some embodiments, is housed or based on a workstation platform 192.

The beam gating system 116 may also include an interface 194 that communicates with the beam controller 168 via a communication channel 196. The communication channel 196 may be an analog or digital cable, or a wireless channel. The beam gating system 116 sends a gating signal via the communication channel 192 to enable or gate the particle emitter 166. Herein, to “enable” the therapy beam emitting system 102 is to cause the particle beam 170 to irradiate the patient 58, while to “gate” the therapy beam emitting system 102 is to prevent the particle beam 170 from irradiating the patient 58. More generally, the term “beam-gating” refers to sending a signal to the beam controller 168 to either pause or resume delivery of the therapy beam 170 as planned. The way such enablement and gating is achieved is system specific. Some systems enable the particle beam 170 by activating an accelerator of the therapy beam emitting system 102 and gate the particle beam 170 by deactivating the accelerator. Other systems leave the accelerator activated and gate the particle emitter 166 by blocking or diverting the particle beam 170 so that the patient 58 is not irradiated.

Functionally, the probe localization system 108 enables tracking of the multiplanar imaging probe 50, 126 in three-dimensional space in order to associate the localization markers 144 with anatomical structures in the multiplanar image 128. The probe localization system 108 enables localization and continuous monitoring of the position localization markers 144 in the room coordinate system 154.

The probe localization system 108 can be used to monitor respiratory motion. The multiplanar imaging probe 50, 126 and/or probe holder 142, being equipped with the localization marker(s) 144 and arranged on the thorax for imaging of the heart, is suitably positioned to follow tidal motion of respiration. As such, the localization marker(s) 144 may act as an external surrogate for respiratory motion. The probe localization system 108 can thereby provide external information about respiratory motion proximate the heart and the target region 146.

In operation, the multiplanar motion management system 118 may be used during the simulation stage 200 (FIG. 5) prior to the treatment delivery and/or during the treatment stage 300 (FIG. 6) where treatment delivery is executed. The multiplanar imaging probe 50, 126 may be secured to the patient 58 with a probe holder 142, for example on the thorax of the patient 58 for an apical or parasternal viewing window. The multiplanar imaging probe 50, 126 is immobilized on the thorax of the patient 58 using the probe holder 142, enabling hands free operation. In some embodiments, signal processing of the beam former of the multiplanar ultrasound probe 50 is utilized to reconstruct an image for visualization.

The multiplanar imaging system 106 may provide continuous visualization of the heart trans-abdominally (through the diaphragm or the liver) or trans-thoracically (between the ribs). The multiplanar imaging probe 50, 126 may be non-parallel, placed in apical position or parasternal position, and/or image long-axis or short-axis heart structures affected by heart motion.

The non-invasive cardiac ablation system 100 also delivers the particle beam 170 to the target region 146 at predetermined phases of certain bodily motion cycles. Such bodily motion cycles may include respiratory and cardiac cycles. The multiplanar imaging system 106 provides the multiplanar images 128 from the real-time imaging to the target motion management system 118. The target motion management system 118 utilizes the multiplanar images 128 to deliver the particle beam 170 at the predetermined respiratory and cardiac phases, for example as described by Garonna et al. The multiplanar images 128 may also include information regarding the properties required of the particle beam 170, which may also be determined by the gating controller 178.

The multiplanar images 128 and cardiac cycle data 158 may be acquired and updated to the target motion management system 118 continuously, providing the operator with a live stream of information. The multiplanar images 128 and cardiac cycle data 158 may also be stored on a storage medium and later analyzed to assess the accuracy of the phase prediction process and, if needed, to adapt the software parameters specific to the patient 58.

Referring to FIG. 5, workflow for a simulation stage 200 is diagrammed according to an embodiment of the disclosure. During the simulation stage 200, all data and anatomical information (e.g., medical images) required for treatment planning are gathered, from which a patient-specific treatment plan is developed. The simulation stage 200 may include a series of processes, each process being designated in FIG. 5 by a reference character beginning with the letter “p”. The processes of the simulation stage 200 may include: a localization system calibration process (p210) which, in some cases, need only be performed intermittently; a subject setup process (p220); a reference imaging process (p230); a planning CT scan process (p240), a contouring process (p250); and a treatment planning process (p260). Various of the processes are depicted as including one or more steps, designated in FIG. 5 by a reference character beginning with the letter “s”.

In some embodiments, the localization calibration process (p210) of the simulation stage 200 takes place in the CT imaging room and includes calibration of the probe localization system 108 using a specifically designed calibration phantom (s211). The calibration phantom step (s211) enables the multiplanar imaging probe 50, 126 to be localized in the room coordinate system 154 (e.g. of the CT imaging room) at later processes and stages. A patient or subject setup process (p220) is executed, wherein the patient 58 is positioned on the CT table following a standard oncological radiotherapy procedure. The subject setup process (p220) may include the use of standard immobilization and patient support devices.

A reference imaging process (p230) is performed, wherein reference multiplanar images are acquired with the multiplanar imaging probe 50, 126 (s231) fixed to the thorax of the patient using the probe holder 142 (s234). During image acquisition, the multiplanar imaging probe 50, 126 may be localized by the probe localization system 108 using the localization markers 144 (s232). In this way, a position of the multiplanar imaging probe 50,126 (referred to herein as the “reference position”) during the acquisition of each reference multiplanar image can be determined with respect to the room coordinate system 154 (s235). The cardiac phase is determined for each of the reference multiplanar images acquired in the reference imaging process p230 (s233). During the acquisition of the reference multiplanar images, skin markers may be placed on the skin of the patient 58 (s236) to aid in repositioning of the multiplanar imaging probe 50, 126 at the treatment stage 300 (FIG. 6). In some embodiments, cardiac cycle data 158 is acquired during the simulation stage (s237). Additionally, the time-resolved position of the localization markers 144 may be used as a respiratory signal (s238).

In some embodiments, the planning CT scan process (p240), the contouring process (p250), and the treatment planning process (p260) of the simulation stage 200 may implement standard oncological radiotherapy procedures. The planning CT scan process (p240) may include planning four-dimensional (three spatial dimensions plus time) computer tomography (4D-CT) scan acquisitions. In some embodiments, a dummy probe is utilized during the acquisition of the planning CT acquisition (s241) to avoid undesirable artifacts on the CT scan generated by metallic parts of the multiplanar imaging probe. The dummy probe may also be localized by the probe localization system 108 using localization markers (s242). Additional cardiac cycle and respiratory data may also be acquired (s244-s247). The 4D-CT scans may be both respiratory- and cardiac-gated. During the contouring process (p250), the radiation target is contoured and, in general, organs at risk near the target region 146 are also contoured. An example relevant contouring process is found at Blanck et al., “Radiosurgery for ventricular tachycardia: preclinical and clinical evidence and study design for a German multi-center multi-platform feasibility trial (RAVENTA),” Clin Res Cardiol 109, 1319-1332 (2020).

In some embodiments, two heartbeat signals are acquired during the planning CT scan process (p240). The planning CT scan is reconstructed using data from a heartbeat signal #1 (s244) which is typically acquired by a first heartbeat sensing system that is connected directly to the CT scanner. Treatment delivery is based on a heartbeat signal #2 (s245) which is acquired the heartbeat sensing system 110 of the I/O management system 114. For ECG systems, the start of a cardiac cycle is usually identified using R-peak information, but different ECG suppliers typically have their own way of identifying the R-peak (e.g., just after or just before the actual peak). Accordingly, the disclosed embodiment includes provisions for acquiring signals from both heartbeat sensing systems simultaneously, to establish a correlation between the R-peak identification of the respective heartbeat sensing systems. Alternatively, the CT scanner may utilize heartbeat signal #2 from the heartbeat sensing system 110 instead of its own heartbeat signal #1, or the I/O management system 114 may utilize heartbeat signal #1 from the CT scanner instead of its own heartbeat signal #2, thereby eliminating the need for correlation.

Likewise, in some embodiments, two respiratory signals RESP #1 and RESP #2 may be acquired during the planning CT scan process (p240). The planning CT scan is reconstructed using data from RESP #1 (s247) which may be acquired by a first localization system available in the CT imaging room (or any other respiration motion surrogate signal). Treatment delivery is based on RESP #2 (s246) which may be acquired by the probe localization system 108 of the I/O management system 114. Each localization system may have peculiarities that require correlation. For example, where localization is performed with optical tracking systems, aspects of one optical tracking system may differ from that of the other optical tracking system. Such aspects include the position of the optical tracking system with respect to the tracked object, the acquisition rate, the acquisition latency. Accordingly, the disclosed embodiment includes acquiring signals from both localization systems simultaneously, to establish a correlation between the respiratory motion as detected by respective localization systems. Alternatively, the CT imaging room may utilize RESP #2 from the probe localization system 108 instead of its own RESP #1, or the I/O management system 114 may utilize RESP #1 from the localization system used in the CT imaging room instead of its own RESP #1, eliminating the need for correlation.

In some embodiments, the treatment planning process (p260) includes determining three input characteristics for the treatment stage 300: (1) a desired location of the treatment isocenter (s261) in the coordinates provided by the room coordinate system 154; (2) a cardiac gating window (s262), given by the treatment plan and determining when the radiation beam should be enabled or disabled based on the heart motion; and (3) a respiratory gating window (s263), given by the treatment plan and determining when the radiation beam should be enabled or disabled based on the respiratory motion. Additionally, the treatment planning process generates the contours for the target and all relevant organs for at least one reference CT.

Referring to FIG. 6, a workflow for a treatment stage 300 is diagrammed according to an embodiment of the disclosure. The treatment stage 300 facilitates delivery of radiation doses accurately to the target while sparing the surrounding normal tissues as much as possible. The treatment stage 300 includes a series of processes, each process being designated in FIG. 6 by a reference character beginning with the letter “p”. The processes may include: a localization system calibration process (p310) which, in some cases, need only be performed intermittently; a subject setup process (p320); a multiplanar probe setup process (p330); a bone alignment process (p340); a heart alignment process (p350); a start treatment process (p360); and a gating process (p370). Various of the processes are depicted as including one or more steps, designated in FIG. 6 by a reference character beginning with the letter “s”.

In some embodiments, the treatment stage 300 starts with the calibration of the probe localization system 108 (p310), again using the calibration phantom (s311). During the subject setup process (p320), the patient 58 is positioned on the table in the same way as in the simulation stage 200. In some embodiments, an initial alignment of skin marks with the treatment room isocenter is performed during the subject setup process (p320), for example, with a laser system available in the treatment room, or with surface imaging systems. The multiplanar imaging probe 50, 126 is positioned and immobilized on the thorax of the patient 58 with the probe holder 142 (s333). To assist with positioning of the multiplanar imaging probe 50, 126 at the same position on the patient 58 as during the simulation stage, skin marks as well as information from the localization markers (s332) may be utilized.

With the multiplanar imaging probe 50, 126 in a fixed position, the patient 58 is aligned using the room coordinate system 154. In some embodiments, the bone alignment process (p340) includes an alignment of the patient 58 based on the bony anatomy, for example using cone-beam CT (s341). During the table alignment step (s342), misalignment is computed based on comparison of bony anatomy from the simulation stage 200 and the treatment stage 300, as identified, for example, with CT, CBCT, or X-ray images. The misalignment is corrected by manipulation of the patient support 172 using the patient positioner 174 to bring the patient 58 into alignment with the simulation orientation. In some embodiments, the correction is performed using the multiplanar motion management system 118 via one of the auxiliary interfaces 120 to control the patient positioning system 104.

During the heart alignment process (p350), live multiplanar images are acquired and registered to the reference multiplanar images that were acquired during the simulation stage 200 (at s235 of p230 of FIG. 5). This rigid registration (s351) is referred to generally as a “live-to-simulation” image registration, and occurs between images of the same data type. For example, when using the multiplanar ultrasound probe 50, the live and simulation images are both ultrasound images, and may be referred to as an “ultrasound-to-ultrasound” or “US-to-US” image registration. The process applies generally regardless of whether the multiplanar images 128 are ultrasound images. In some embodiments, the ultrasound-to-ultrasound image registration serves as a final verification of the patient setup. For the table fine tuning step (s353), misalignment is computed based on comparison of soft tissue contrast of the heart from the simulation stage 200 and the treatment stage 300, as identified with the multiplanar images 128. The necessary translations and rotations to correct the misalignment are performed with the positioner system 174, optionally using the multiplanar motion management system 118.

At the start of the treatment and gating processes (p360 and p370), live multiplanar images are continuously acquired and processed in real time or near real time (s371), with the probe localization system 108 simultaneously tracking the multiplanar imaging probe 50, 126 (s372). The cardiac phase is determined live for each of the reference multiplanar images acquired in the reference imaging process p230 (s233). Based on the live multiplanar images, four different outputs are generated: (1) heart displacement information for drift monitoring (s374); (2) a respiratory gating signal (s375); (3) a cardiac gating signal (s376) (e.g., such as disclosed in Garonna et al.); and (4) associated warnings and interlocks (s377), such as patient physiological changes that may require medical attention and patient physiological changes that require treatment interruption.

Referring to FIGS. 7 through 9, a patient setup method 400 that enhances alignment of the patient to the correct position in the room coordinate system 154 is depicted according to an embodiment of the disclosure. The patient setup method assists in setting up the patient 58 in the same position and orientation in the treatment stage 300 as in the simulation stage 200 by complementing the bony alignment setup with the heart alignment setup using multiplanar imaging. The patient setup method 400 may be accomplished during the heart alignment process (p350) of the treatment stage 300. Aspects of the method are outlined below.

At the simulation stage 200, during localization of the multiplanar imaging probe 50, 126 (FIG. 5), a simulation stage image sequence SIM is acquired over a duration of one or more heartbeats and one or more respiration cycles. Similarly, at the treatment stage 300, during localization of the multiplanar imaging probe 50, 126 (FIG. 6), a treatment stage image sequence TX is acquired over a duration of one or more heartbeats and respiration cycles.

From both the SIM and the TX sequences, the multiplanar images 128 belonging to a particular combination of cardiac and respiration motion phases are extracted (step 400a at FIG. 7), and the simulation stage reference pixel coordinates 156′ and treatment stage reference pixel coordinates 156 are defined. For example, the images that correlate with an end-expiration respiratory phase and an end-systole cardiac phase could be extracted from each sequence. In this way, the cardiac and respiratory motion are in a same physiological state for the extracted images from the SIM and TX sequences. Determination of the known respiratory state may be performed by the gating controller. Such known respiratory state includes but is not limited to a breath hold condition or an end-expiration condition. Also, determination of the end-systole cardiac phase or other preferred cardiac phase may be determined by the gating controller.

A coarse rigid registration is performed at step 400b (FIG. 8). The coarse rigid registration overlaps images 402 and 402′ from the SIM and TX sequences. The coarse rigid registration is based on the position of the effective probe volume 56 at the time of image acquisition (step 400b at FIG. 8). The coarse rigid registration 400b is performed without regard to the content of the SIM and TX images 402 and 402′.

A fine rigid registration is also performed, based on the content of the SIM and TX images 402 and 402′ (step 400c at FIG. 9). The combination of the coarse and the fine registrations quantifies the translational and rotational displacement of the heart at treatment stage 300 relative to the simulation stage 200. Accordingly, the patient setup method (processes p320, p340, and p350 of the treatment stage 300 of FIG. 6) may provide translational and rotational instructions to the patient positioning system 104 for displacing the patient support 172 of the non-invasive cardiac ablation system 100 upon which the patient 58 is positioned, in order to correctly align the heart of the patient 58 with respect to the simulation stage setup (steps s342 and S353 of FIG. 6). The basic principles of coarse and fine rigid registration are disclosed by Khalil et al., “An Overview on Image Registration Techniques for Cardiac Diagnosis and Treatment,” Cardiology research and practice, 2018 (2018), and by Che et al., “Ultrasound registration: A review,” Methods, 115, 128-143 (2017).

The technique described above can also be used to monitor the heart displacement or “drift” in reference pixel coordinates 156 during the treatment stage 300. That is, the heart drift may also be measured with respect to the SIM images 402. The information can be visualized on the GUI 182 to enable interruption the treatment as the operator may deem necessary. In some embodiments, based on the inputs given by the operator prior to treatment delivery, the non-invasive cardiac ablation system 100 can automatically trigger warnings and/or interlocks and interrupt the particle beam 170 if, for example, the heart moves outside of the planned target margin (accounting for motion uncertainties) for too long.

In some embodiments, external and internal information is combined to monitor the motion of the heart due to respiration. Placement of the multiplanar imaging probe 50, 126 and attendant localization marker(s) 144 on the thorax of the patient (s332, s333 of FIG. 6) provides the external information, as discussed above. The multiplanar images 128 provides the internal information about the motion of the heart resulting from respiration. In fact, the multiplanar images 128 contain information about the motion of the heart as well as the motion of the multiplanar imaging probe 50, 126 in the reference pixel coordinates 156.

A combination of both the external and the internal information provides an accurate representation of the heart motion, whereas relying on external information enables only an inaccurate inference of the heart motion based on the movement of the surrounding anatomical structures, such as the lungs. The combination of internal and external information for respiratory motion management is an improvement over conventional systems and techniques, which are often limited to external information provided by an external surrogate.

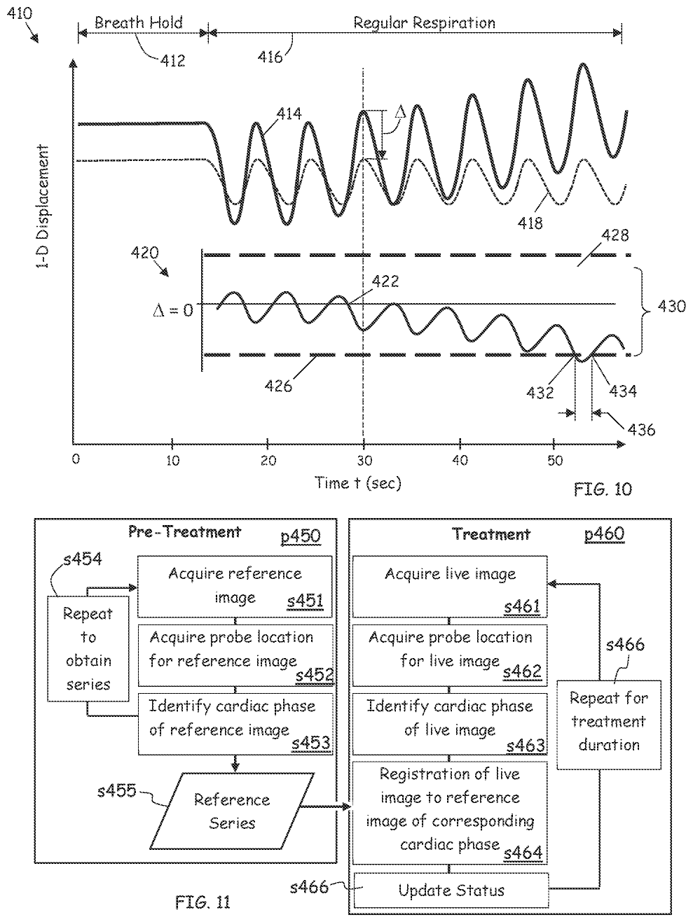

Referring to FIG. 10, various aspects of respiratory motion information are discussed according to an embodiment of the disclosure. The multiplanar images 128 inherently capture both the displacement of the heart relative to the multiplanar imaging probe 50, 126 (resulting from an anatomical structure displacement inside the thorax) and the displacement of the multiplanar imaging probe 50, 126 itself (resulting from thorax expansion and contraction). These combined displacements can be isolated and extracted from the multiplanar images 128 by a rigid registration process. The probe localization enables dissociation of the two contributions. The registration process enables determination of the heart displacement.

In some embodiments, at the start of the treatment (e.g., during the start treatment process p360 of FIG. 6), a series of reference multiplanar images of the beating heart of the patient are acquired when the patient is in a known respiratory state. The patient may be asked, for example, to hold his or her breath over one or more cardiac cycles, as depicted in a breath hold condition 412. For each reference multiplanar image in this breath hold or reference sequence 412, motion due to respiration is absent and the cyclical motion attributed to cardiac motion may be extracted in terms of cardiac phase based on the cardiac cycle data 158 or as described in Garonna et al. Alternatively, instead of generating the reference multiplanar images at the start of the treatment, the SIM images 402 generated during the simulation stage 200 (acquired at s235 of p230 of FIG. 5) may be used.

During the treatment stage 300, a series of live multiplanar images is acquired. A rigid registration of each live multiplanar image to a reference multiplanar image of corresponding cardiac phase is performed. For example, if the cardiac phase for a given live multiplanar image is identified as being 0.3, that live multiplanar image is registered to a reference multiplanar image that corresponds to or represents the same cardiac phase of 0.3. This rigid (live-to-simulation) image registration may be similar to the steps for the TX to SIM registration described attendant to FIGS. 7 through 9 and include a fine and a coarse registration.

During treatment, the patient is able to breathe normally, as indicated by a fine rigid registration time trace 414 during a regular respiration condition 416. The fine registration time trace 414 of the live-to-simulation images depicts the displacement of the heart with respect to the probe because the images by themselves provide no indication of the spatial position of the probe volume 56 during the acquisitions. A localization marker time trace 418 presenting the displacement of the localization markers 144 during the regular respiration condition 416 is also presented in FIG. 10. In contrast, the localization marker time trace 418 provides only an indication of the displacement in position of the probe volume with respect to the room coordinates. The live multiplanar images acquired during regular respiration 416 are registered to the reference multiplanar image of corresponding cardiac phase. Because of the cardiac phase correlation of the reference and live multiplanar images, any relative displacement due to cardiac activity is effectively cancelled by the rigid registration, such that any displacement indication after the rigid registration of the live-to-simulation (ultrasound-to-ultrasound) images is due primarily to respiratory motion and/or heart drift relative to the multiplanar imaging probe 50, 126. The motion of the heart due to respiration can thus be inferred from the difference between the rigid registration and localization time traces 414 and 418.

Inference of the heart motion due to respiration is accomplished by a full (coarse and fine) registration, also illustrated in FIG. 10. The procedure for the full registration is as follows: A difference A between the fine rigid registration time trace 414 and localization (coarse registration) time trace 418 is illustrated at a time t of 30 seconds in graph 410. The difference A may be in reference to the registration time trace 414, in which case the value of A illustrated at t=30 seconds is negative or inverted (represented by the down arrow in graph 410). A subplot 420 presenting a difference time trace 422 of differences A as a function of time is also provided in FIG. 10. The cyclical aspect of the difference time trace 422 represents the motion of the heart due to respiration, and provides a reliable and dynamic indication of the respiratory cycle for purposes of respiratory gating or motion monitoring.

Detection of heart drift is also depicted in subplot 420. Generally, the upward migration of the fine registration time trace 414 relative to the localization time trace 418 in graph 410 is indicative of heart drift. The heart drift is indicated in subplot 420 by a downward migration of the difference time trace 422. The opposite directions of the migration of time traces 414 and 422 is attributed to the inverted magnitude of the difference A. Over the initial respiratory cycle(s) of the regular respiration phase 416, a median datum line 424 may be established that represents the difference A of zero. An operating band 430 is bounded by lower and upper threshold values 426 and 428 of the difference A on the subplot 420. The operating band represents an allowable range of differences A within which the non-invasive cardiac ablation system 100 may operate. The difference time trace 422 is depicted as exceeding the operating band 430 at the lower threshold value 426 at an egress point 432 and returning to within the operating band 430 at a reentrance point 434. As such, there is a breach 436 of the operating band 430 that exists between the egress and reentrance points 432 and 434.

In some embodiments, the beam gating system 116 is configured to gate or disable delivery of the therapy beam 170 if the difference A is outside the operating band 430. The beam gating system 116 may also be configured to enable delivery of the therapy beam 170 if the difference A reenters the operating band 430. In some embodiments, the beam gating system 116 is configured to gate delivery of the therapy beam 170 only if the breach 436 exceeds a predetermined time duration. Likewise, the beam gating system 116 is configured to enable delivery of the therapy beam 170 only if the reentry into the operating band exceeds a predetermined time duration. In some embodiments, the upper and lower threshold values 426 and 428 is entered by the system operator, for example using the control console 180.

The graph 410 of FIG. 10 is included for purposes of illustration and presents one-dimensional displacement data along a single coordinate. The skilled artisan will recognize, in view of this disclosure, that the multiplanar imaging probe 50, 126 provides information in three dimensions, and therefore can provide displacement information in three dimensions.

The time traces 414 and 418 have the same temporal pattern, as both are a consequence of the patient respiration. As such, the localization marker time trace 418 may provide a redundancy that serves as a check to the live-to-simulation image registrations. Also, in case the live-to-simulation (e.g., US-to-US) registration fails, monitoring the localization marker(s) 144 localization can provide a backup for determining respiratory phase. In addition, the information provided by the localization marker may provide extra information which can be used to further understand how the anatomical structures around the heart are positioned.

Referring to FIG. 11, a method for extracting respiratory motion information from the multiplanar images 128 based on the aspects of respiratory motion discussed at FIG. 10 is depicted according to an embodiment of the disclosure. A reference multiplanar image of a beating heart within a patient is acquired with a multiplanar imaging probe (s451); concurrently acquiring a reference position of the imaging probe for the reference multiplanar image of step s451 (s452); identifying a cardiac phase for the reference multiplanar image of step s451 (s453); and repeating steps s451 through s453 to acquire a series of reference multiplanar images and the corresponding probe locations and cardiac phases (s454). The series of reference multiplanar images is stored for access during the treatment process p460, for example in the computer-readable storage medium of the gating controller 178.

The treatment process 460 is subsequently executed: acquiring a live multiplanar image with the multiplanar imaging probe during beam therapy treatment of the patient (s466); concurrently acquiring a live position of the multiplanar imaging probe (s467); identifying a cardiac phase of the live multiplanar image (s467); performing a rigid registration of the live multiplanar image and the live position to a corresponding reference image of the reference series and a corresponding reference position associated with the corresponding reference image acquired at step s455, the corresponding reference image being representative of the cardiac phase of the live multiplanar image. The rigid registration provides a live heart position of the beating heart during the beam therapy treatment of the patient relative to a three-dimensional coordinate system to provide a live displacement of the beating heart relative to the reference position. In some embodiments, a status condition based on a comparison of said displacement with a predetermined threshold value is generated for updating by the beam gating system 116. Steps s461 through 466 are repeated for the duration of the external beam therapy treatment.

In some embodiments, steps of the various methods disclosed herein are provided in the form of instructions provided on a tangible, non-transitory medium, such as on a printed document, compact disc, or flash drive. Non-limiting examples of a tangible, non-transitory medium include a paper document and computer-readable media including compact disc and magnetic storage devices (e.g., hard disk, flash drive, cartridge, floppy drive). The computer-readable media may be local or accessible over the internet. The instructions may be complete on a single medium, or divided among two or more media. For example, some of the instructions may be written on a paper document that instruct the user to access one or more of the steps of the method over the internet, the internet-accessible steps being stored on a computer-readable medium or media.

Each of the additional figures and methods disclosed herein can be used separately, or in conjunction with other features and methods, to provide improved devices and methods for making and using the same. Therefore, combinations of features and methods disclosed herein may not be necessary to practice the disclosure in its broadest sense and are instead disclosed merely to particularly describe representative and preferred embodiments.

Various modifications to the embodiments may be apparent to one of skill in the art upon reading this disclosure. For example, persons of ordinary skill in the relevant arts will recognize that the various features described for the different embodiments can be suitably combined, un-combined, and re-combined with other features, alone, or in different combinations. Likewise, the various features described above should all be regarded as example embodiments, rather than limitations to the scope or spirit of the disclosure.

Persons of ordinary skill in the relevant arts will recognize that various embodiments can comprise fewer features than illustrated in any individual embodiment described above. The embodiments described herein are not meant to be an exhaustive presentation of the ways in which the various features may be combined. Accordingly, the embodiments are not mutually exclusive combinations of features; rather, the claims can comprise a combination of different individual features selected from different individual embodiments, as understood by persons of ordinary skill in the art.

The following documents are hereby incorporated by reference herein in their entirety: Topolnjak et al., “Image-guided radiotherapy for left-sided breast cancer patients: geometrical uncertainty of the heart,” International Journal of Radiation Oncology* Biology* Physics, 82(4), e647-e655 (2012); Kincaid Jr, et al., “Investigation of gated cone-beam CT to reduce respiratory motion blurring,” Medical physics, 40(4), 041717 (2013); Nankali et al., “Geometric and dosimetric comparison of four intrafraction motion adaptation strategies for stereotactic liver radiotherapy,” Physics in Medicine & Biology, 63(14), 145010 (2018); Fayad et al., “Correlation of respiratory motion between external patient surface and internal anatomical landmarks,” Medical physics, 38(6Part1), 3157-3164 (2011); Robinson et al., “An evaluation of the Clarity 3D ultrasound system for prostate localization,” Journal of applied clinical medical physics, 13(4), 100-112 (2012); De Luca et al., “The 2014 liver ultrasound tracking benchmark,” Physics in Medicine & Biology, 60(14), 5571 (2015); Lachaine et al., “Intrafractional prostate motion management with the Clarity Autoscan system,” Medical physics international, 1 (2013); Ipsen et al., “Target tracking accuracy and latency with different 4D ultrasound systems—a robotic phantom study,” Current Directions in Biomedical Engineering, 6(1) (2020); Queiros et al., “Fast left ventricle tracking using localized anatomical affine optical flow,” International journal for numerical methods in biomedical engineering, 33(11), e2871 (2017); Roujol et al., “Characterization of respiratory and cardiac motion from electro-anatomical mapping data for improved fusion of MRI to left ventricular electrograms,” PloS one, 8(11), e78852 (2013); Baker et al., “Prostate displacement during transabdominal ultrasound image-guided radiotherapy assessed by real-time four-dimensional transperineal monitoring,” Acta Oncologica, 54(9), 1508-1514 (2015); Khalil et al., “An Overview on Image Registration Techniques for Cardiac Diagnosis and Treatment,” Cardiology research and practice, 2018 (2018); Che et al., “Ultrasound registration: A review,” Methods, 115, 128-143 (2017); Hoogeman et al., “Clinical accuracy of the respiratory tumor tracking system of the cyberknife: assessment by analysis of log files,” International Journal of Radiation Oncology* Biology* Physics, 74(1), 297-303 (2009); Depuydt et al., “Treating patients with real-time tumor tracking using the Vero gimbaled linac system: implementation and first review,” Radiotherapy and Oncology, 112(3), 343-351 (2014); Keall et al., “The management of respiratory motion in radiation oncology report of AAPM Task Group 76a” (2006); Blanck et al., “Radiosurgery for ventricular tachycardia: preclinical and clinical evidence and study design for a German multi-center multi-platform feasibility trial (RAVENTA),” Clin Res Cardiol 109, 1319-1332 (2020) Zei, et al. “Ablative Radiotherapy as a Noninvasive Alternative to Catheter Ablation for Cardiac Arrhythmias” Curr Cardiol Rep (2017); van der Ree et al., “Cardiac Radioablation—a Systematic Review”, Heart Rhythm (2020); Graeff et al., “Noninvasive cardiac arrhythmia ablation with particle beams”, Med Phys 45 (11) (2018); O'Shea et al., “Review of ultrasound image guidance in external beam radiotherapy part II: intra-fraction motion management and novel applications”, Physics in Medicine & Biology, v61(8) (2016); Fontanarosa et al., “Review of ultrasound image guidance in external beam radiotherapy: I. Treatment planning and inter-fraction motion management”, Physics in Medicine & Biology, v60(3), R77 (2015); Bertholet et al., “Real-time intrafraction motion monitoring in external beam radiotherapy”, Physics in Medicine 64 (15) (2019); Ipsen, “See what you treat: 4d ultrasound imaging for real time motion compensation in the liver”, PhD Thesis University of Luebeck (2019); Ipsen et al, “Radiotherapy beyond cancer: Target localization in real-time MRI and treatment planning for cardiac radiosurgery”, Med Phys 41 (12) (2014); Poon et al., “Technical Note: Cardiac synchronized volumetric modulated arc therapy for stereotactic arrhythmia radioablation—Proof of principle”, Med Phys 47 (8) 2020.

Any incorporation by reference of documents herein is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Unless indicated otherwise, references to “embodiment(s)”, “disclosure”, “present disclosure”, “embodiment(s) of the disclosure”, “disclosed embodiment(s)”, and the like contained herein refer to the specification (text, including the claims, and figures) of this patent application that are not admitted prior art.

For purposes of interpreting the claims, it is expressly intended that the provisions of 35 U.S.C. 112(f) are not to be invoked unless the specific terms “means for” or “step for” are recited in the respective claim.

1. A method for detecting displacement of a heart within a patient during an external beam therapy treatment, comprising:
configuring a tangible, non-transitory medium with instructions, said instructions comprising:
(a) acquiring a series of reference multiplanar images of a beating heart within a patient with a multiplanar imaging probe;
(b) during step (a), acquiring a reference position of said imaging probe for each of the reference multiplanar images of said reference series of multiplanar images;
(c) identifying a cardiac phase for each reference multiplanar image of said reference series of multiplanar images;
(d) acquiring a live multiplanar image with said multiplanar imaging probe during beam therapy treatment of said patient;
(e) during step (d), acquiring a live position of said multiplanar imaging probe;
(f) identifying a cardiac phase of said live multiplanar image; and
(g) performing a rigid registration of said live multiplanar image and said live position to a corresponding reference image of said reference series of multiplanar images and a corresponding reference positions associated with said corresponding reference image, said corresponding reference image being representative of said cardiac phase of said live multiplanar image, said rigid registration providing a live heart position of said beating heart during said beam therapy treatment of said patient relative to a three-dimensional coordinate system to provide a live displacement of said beating heart between relative to said reference position.

2. The method of claim 1, wherein said instructions in the step of configuring includes generating a status condition based on a comparison of said displacement with a predetermined threshold value.

3. The method of claim 1, wherein said series of multiplanar images acquired in step (a) are acquired with said patient in a known respiratory state.

4. The method of claim 3, wherein said known respiratory state is an end-expiration respiratory phase of said patient.

5. The method of any one of claims 1-4, comprising providing said tangible, non-transitory medium.

6. The method of any one of claims 1-4, comprising providing a multiplanar imaging probe for acquiring the multiplanar images of steps (a) and (d).

7. The method of any one of claims 1-4, comprising providing a probe localization system for acquiring the positions of said imaging probe at steps (b) and (e).

8. The method of any one of claims 1-4, comprising providing a gating controller for accessing said tangible, non-transitory medium.

9. A method for determining a position of cardiac structures in a known three-dimensional coordinate system, comprising:
providing a multiplanar imaging probe and a localization system for tracking said multiplanar imaging probe in three-dimensional space;
providing instructions on a tangible, non-transitory medium, said instructions comprising:
acquiring a first sequence of multiplanar images of a heart with said multiplanar imaging probe over a duration of multiple heartbeats while said patient is in a known respiratory state, said heart being at a first position;
during the step of acquiring said first sequence, localizing said multiplanar imaging probe with said localization system to define a first set of room coordinates at said first position;
acquiring a second sequence of multiplanar images of said heart with said multiplanar imaging probe over a duration of multiple heartbeats while said patient is in said known respiratory state, said heart being at a second position;
during the step of acquiring said second sequence, localizing said multiplanar imaging probe with said localization system to define a second set of room coordinates at said second position;
identifying a first set of representative multiplanar images from said first sequence of multiplanar images and a second set of representative multiplanar images from said second sequence of multiplanar images, said first set of representative multiplanar images and said second set of representative multiplanar images corresponding to a known combination of a cardiac motion phase and a respiratory motion phase;
aligning said second set of room coordinates with said first set of room coordinates; and
quantifying a translational displacement and a rotational displacement of said second set of representative multiplanar images relative to said first set of representative multiplanar images within the aligned first and second set of room coordinates.

10. The method of claim 9, wherein said known combination of cardiac motion phase and respiratory motion in the step of identifying correspond with an end-expiration of said respiratory motion phase.

11. The method of claim 9, wherein said instructions in the step of providing instructions include displacing a patient support in accordance with said translational displacement and said rotational displacement.

12. The method of claim 9, wherein said instructions in the step of providing instructions include instructing said patient to breath hold over at least one cardiac cycle during the step of acquiring said first sequence of multiplanar images to effect said known respiratory state.

13. The method of claim 9, wherein the step of acquiring said first sequence of multiplanar images of said instructions is performed at an end-expiration respiratory phase to effect said known respiratory state.

14. A method for enabling and gating a therapy beam, comprising:
coupling a multiplanar imaging probe and a probe localization system to a gating controller, said gating controller including a set of instructions on a non-transitory computer readable medium for execution in real time, said instructions including:
(a) acquiring a series of reference multiplanar images of a beating heart within a patient with a multiplanar imaging probe;
(b) during step (a), acquiring a reference position of said imaging probe for each of the reference multiplanar images of said reference series of multiplanar images;
(c) identifying a cardiac phase for each reference multiplanar image of said reference series of multiplanar images;
(d) acquiring a live multiplanar image with said multiplanar imaging probe during beam therapy treatment of said patient;
(e) during step (d), acquiring a live position of said multiplanar imaging probe;
(f) identifying a cardiac phase of said live multiplanar image; and
(g) performing a rigid registration of said live multiplanar image and said live position to a corresponding reference image of said reference series of multiplanar images and a corresponding reference positions associated with said corresponding reference image, said corresponding reference image being representative of said cardiac phase of said live multiplanar image, said rigid registration providing a live heart position of said beating heart during said beam therapy treatment of said patient relative to a three-dimensional coordinate system to provide a live displacement of said beating heart between relative to said reference position.

15. The method of claim 14, wherein said instructions of said gating controller includes generating a status condition based on a comparison of said displacement with a predetermined threshold value.

16. The method of claim 15, comprising configuring said non-transitory computer readable medium with said instructions.

17. A method for inferring displacement of a heart of a patient for beam therapy treatment, comprising:
(a) acquiring a series of reference multiplanar images of a beating heart within a patient with a multiplanar imaging probe;
(b) during step (a), acquiring a reference position of said imaging probe for each of the reference multiplanar images of said reference series of multiplanar images;
(c) identifying a cardiac phase for each reference multiplanar image of said reference series of multiplanar images;
(d) acquiring a live multiplanar image with said multiplanar imaging probe during beam therapy treatment of said patient;
(e) during step (d), acquiring a live position of said multiplanar imaging probe;
(f) identifying a cardiac phase of said live multiplanar image; and
(g) performing a rigid registration of said live multiplanar image and said live position to a corresponding reference image of said reference series of multiplanar images and a corresponding reference positions associated with said corresponding reference image, said corresponding reference image being representative of said cardiac phase of said live multiplanar image, said rigid registration providing a live heart position of said beating heart during said beam therapy treatment of said patient relative to a three-dimensional coordinate system to provide a live displacement of said beating heart between relative to said reference position.

18. The method of claim 17, comprising generating a status condition based on a comparison of said displacement with a predetermined threshold value.

19. The method of claim 18, comprising affixing said multiplanar imaging probe to a thorax of said patient for use during execution of a reference simulation.

20. The method of claim 18, comprising affixing said multiplanar imaging probe to a thorax of said patient for use during execution of a simulation stage.

21. The method of claim 18, comprising instructing said patient to breath hold over at least one cardiac cycle to effect said known respiratory state.

22. The method of claim 18, wherein said known respiratory state is an end-expiration respiratory phase.

23. The method of claim 18, wherein said reference series of multiplanar images is acquired at the start of a treatment process.

24. The method of claim 18, wherein said reference series of multiplanar images is acquired during a simulation stage prior to a treatment stage.

25. A method for inferring displacement of the heart resulting from respiratory motion from a multiplanar imaging probe mounted to a thorax of a patient, comprising:
providing a multiplanar imaging probe;
providing instructions on a tangible, non-transitory medium, said instructions comprising:
acquiring a reference series of multiplanar images with said multiplanar imaging probe, said multiplanar imaging probe being affixed to a thorax of a patient;
identifying a cardiac phase for each multiplanar image of said reference series of multiplanar images;
acquiring a live multiplanar image with said multiplanar imaging probe during irradiation treatment of said patient, said multiplanar imaging probe being affixed to said thorax of said patient;
identifying a cardiac phase for said live multiplanar image; and
performing a rigid registration of said live multiplanar image to a corresponding image of said reference series of multiplanar images, said corresponding image of said reference series being representative of said cardiac phase of said live multiplanar image.

26. A multiplanar motion management system for non-invasive cardiac ablation, comprising:
a multiplanar imaging probe configured to acquire live multiplanar images within a probe volume, said multiplanar images being non-parallel;
a probe localization system configured to acquire positional data of said multiplanar imaging probe;
an input/output (I/O) management system configured to concurrently receive said live multiplanar images and said positional data, and to establish acquisition times for said multiplanar images and for said positional data;
a gating system configured to receive and process said live multiplanar images, said positional data, and said acquisition times, said gating system including a processor configured to:
identify a cardiac phase corresponding to said live multiplanar images;
define a set of room coordinates from said positional data that closest corresponds in time to said live multiplanar images; and
perform a rigid registration of said live multiplanar images to reference multiplanar images, said reference multiplanar images being representative of said cardiac phase.

27. The multiplanar motion management system of claim 26, comprising a heartbeat sensing system for generating cardiac cycle data, wherein:
said heartbeat sensing system is configured to generate a trigger signal based on said cardiac cycle data;
said I/O management system is configured to receive said trigger signal concurrently with said multiplanar images and said positional data, and to generate trigger signal information that includes an acquisition time corresponding to said trigger signal; and
said gating system is configured to receive and process said trigger signal information for identification of a start of a cardiac cycle.

28. The multiplanar motion management system of claim 27, wherein said gating system is configured to utilize said trigger signal information to confirm said cardiac phase.

29. The multiplanar motion management system of claim 27, wherein said heartbeat sensing system is an electrocardiogram (ECG) system and said cardiac cycle data is generated from an electrocardio signal.

30. The multiplanar motion management system of claim 26, wherein said positional data is relative to a room coordinate system.

31. The multiplanar motion management system of claim 26, wherein said live multiplanar images and said reference multiplanar images are of a same data type.

32.-35. (canceled)

36. The multiplanar motion management system of claim 26, wherein said positional data localizes a location and orientation of said multiplanar imaging probe in three-dimensional space.

37. The multiplanar motion management system of claim 26, wherein said multiplanar imaging probe is configured to acquire said multiplanar images simultaneously.

38. The multiplanar motion management system of claim 26, wherein said multiplanar imaging probe includes a geometry configured to mount on the thorax of a patient.

39. The multiplanar motion management system of claim 26, wherein said multiplanar imaging probe acquires said multiplanar images at a rate that is in a range of 20 Hz to 50 Hz inclusive.

40. The multiplanar motion management system of any one of claims 28-31 or any one of claims 36-39, wherein said multiplanar imaging probe is a multiplanar ultrasonic probe and said reference multiplanar images are ultrasonic images.
